(19) United States
(12) Patent Application Publication
(10) Pub. No.: US 2007/0253993 A1
(43) Pub. Date: Nov. 1, 2007

(54) Climate, respectively ventilation channel

(76) Inventor: Ina Bruer, Schofferstadt (DE)

Correspondence Address: SHERIDAN ROSS PC, 1560 BROADWAY, SUITE 1200, DENVER, CO, 80202, US

(21) Appl. No.: 10/574,987

(22) PCT Filed: Oct. 4, 2004
(86) PCT No.: PCT/EP04/11064

§ 371 Date: Mar. 15, 2007

(30) Foreign Application Priority Data
Oct. 6, 2003 (EP) 03022613.8
Jan. 7, 2004 (FR) 0400084

(51) Int. Cl. A61F 13/00; B32B 27/04

(52) U.S. Cl. 424/422; 442/123

(57) ABSTRACT

At a climate, respectively ventilating channel, for the inner-and/or outer lining, insulating elements with a temperature resistance to observe normative demands of the fire resistance categories are being used, whose fiber composition features an alkali/earth alkali mass relation of <1, whose fiber structure is determined by an average geometric fiber diameter of ≦4 μm, whose gross density is in the range of 20 to 120 kg/m³ and whose binding agent portion in the form of a plate, is within the range of 4,5 to 7 weight % or in the form of a wire mesh mat within the range of 0,5 to 1 weight %.

The present invention relates to a climate, respectively ventilation channel, according to the preamble of claim 1.

Such ventilation channels are normally lined internally and/or externally for insulating purposes, and the lining usually is composed of mineral wool. In this case, the internal insulation is normally applicable for heat and sound insulation, whilst the outer insulation is usually designed for fire protection.

The internal insulation of said climate, respectively ventilation channel, is exposed in the flowing conducting fluid, such as air, to eventually high temperature levels and—especially in the cases of flow speeds of up to 30 m/s—to high forces resulting from pulsation and twirling. Critical points for this application of force are, on one side, junction points, located transversally to the flow direction between insulating elements and, on the other side, attachment points by means of retaining disks on the insulating substance surface. At the junction points, there is a trend of the flux to penetrate into the junction area, loosening the fiber connections at those points, i.e. features a trend to suspend a lamination provided at that point. At the retaining disks, there are forcibly asperities of the flux marginal areas, caused by compressed insulating material, which result in force being exerted, due to depositions resulting from twirling action or similar occurrences.

As a consequence, for example in the case of the internal insulation, the resistance of the insulating material, i.e. of the fiber connection, forming said insulating material, and elements attached thereon, such as laminated sections, are of special significance. In the area of the retaining disks, a high resistance results in a reduction of the so called “mattress effect”, which appears when the retaining disks deeply penetrate into the surface of the insulating material, in order to be able to transfer the required retaining forces.

For the internal insulation of ventilation channels, mostly glass wool material is being utilized, which usually features fine and long fibers, and in the case of corresponding binding agent content, offers a relatively high rigidity and firmness. Such products normally feature a λ-arithmetic value according to DIN 18165, located between 30 and 40 mW/mK, with a relatively low gross density below 25 kg/m³. As binding agent, usually melamine resin is being used in view of the question of combustibility (for example, building material category A1/A2), whist normally with mineral fiber products, for price reasons, preferably phenol-formaldehyde resin is being utilized.

The demands formulated in the case of the outer insulation of climate, respectively ventilation lines, which are of an essential nature for fire protection purposes, especially refer to the fact that the ventilation channel remains physically preserved beyond a certain time span, in case of fire. In addition, in the cases of wall passages, care should be taken that no quick passage of fire from one room to another room takes place, with excessively high temperature increase in the contiguous room.

The fire protection demands from such systems are therefore graded in the so called fire resistance categories or similar units. Fire resistance category L30 means, for example, that the line construction, under standardized test conditions, is capable of resisting to a fire load, i.e. exposure, during 30 minutes. According to the usage, for example the fire resistance categories L30, L60 or L90 are required.

Especially to obtain higher fire resistance categories, as insulating material for such conducting channels, the use of rock wool is necessary, whose point of fusion according to DIN 4102, Part 17, is placed at 1.000° C. and which, therefore, compared to glass wool, distinguishes itself by a higher temperature resistance rate. Such rock wool is commonly produced in the so called nozzle blowing process or with external centrifugation, such as the so called cascade centrifuging process. In this case, relatively coarse fibers are produced with an average geometric diameter above 4 to 12 μm of relatively lower length. As binding agent, normally phenol-formaldehyde resin is being used. As a result of the production, also a considerable portion of non-fibrillated material is provided in the product in the form of so called “beads”, with a particle size of at least 50 μm, participating of the weight, but not of the desired insulating effect. The normal portion of “beads” is found, in this case, between 10 and 30 weight %, meaning the portion of unfiberized material, therefore coarser fiber components.

Based on the coarser fiber structure vis-à-vis glass wool, conventional rock wool, with identical λ-arithmetic values and identical insulating thickness, features a significantly higher gross density and, therefore, also higher weight. Also the conventional rock wool, with identical λ-arithmetic value and identical gross density like conventional glass wool, offers a significantly higher insulating thickness and, therefore, an essentially larger volume.

A characteristic feature of differentiation between glass and rock wool as subgroups of the species mineral wool, consists in an alkali/earth alkali relation of the composition, which in the case of rock wool is <1 and in the case of glass wool >1. This means that rock wool has a high portion of CaO+MgO, for example of 20 to 30 weight % and a relatively low portion of Na₂0+k₂0, for example of approximately 5 weight %. Glass wool, on its turn, normally contains earth alkaline components of at least, approximately, 10 weight % and alkali components above 15 weight %. These figures represent especially non-characteristic and non-biopersistent, i.e. biosoluble compositions.

Mineral fibers produced with internal centrifugation according to the centrifuging basket process, with a comparably high temperature resistance, are known from documents EP 0 551 476, from EP 0583 792, from WO 94/04468 as well as U.S. Pat. No. 6,284,684, to which express reference is now being made with a view to providing further details.

Based on this background, the object of the invention consists in creating a climate, respectively ventilation channel, which is comparably built with thin walls and/or light weight, fulfilling in the same way the normative demands related to sound, heat and fire protection. Especially the insulating elements, provided for the internal and/or external lining, should be adequate for this performance, being also sufficiently resistant and stable, especially in order to be in a condition to safely resist—for extended operational periods—to the loads, resulting from the flowing medium.

According to the invention, for a climate, respectively ventilation channel, this task is being solved with the features of the marking part of claim 1.

According to determination by the invention, this is being attained by the controlled cooperation of different factors, i.e. configuration of the fibers according to an average geometric fiber diameter of ≦4 μm and adjustment of the gross density of the mineral fibers according to fire resistance class in a range of 20 to 120 kg/m³, as well as an addition of binding agent for hardening of the mineral fibers, in the form of a plate of 4%, particularly 4,5% to 7 weight %, relative to the fiber mass of said insulating elements, or in the form of a wire mesh mat above 0,5 to 1 weight %. Additionally, the composition of the mineral fibers of the insulating element should feature an alkali/earth alkali mass relation of <1. Due to a finely structured mineral fiber with an average geometric fiber diameter of ≦4 μm, a fiber structure results, at which, with similar gross density as in the case of conventional rock wool fibers, essentially more fibers are provided in the structure and, therefore, also a large number of crossing points for the fiber connection. With similar application of binding agent as with conventional rock wool, in view of the larger number of crossing points and concentration of the binding agent at these points, there will be an essential reduction of the portion of binding agent which does not contribute to a binding effect, resulting in a fiber connection, which offers a comparably more rigid configuration of a hardened fiber connection. Also, as a result of the finer fiber structure of the insulating elements according to the invention, this may be configured comparably lighter, with a gross density according to the normative fire resistance category or similar in the range of 20 to 120 kg/m³ and, therefore, compared to insulating elements of conventional rock wool, which usually feature gross densities between 45 and 180 kg/m³. In this case, with identical absolute organic fire cargo, i.e. binding agent application, a correspondingly large relative binding agent portion may be adjusted, which results in that the plate comparably will be essentially more rigid. On the other side, with the insulating plate according to the invention, a given rigidity and stability may also be attained with a comparably lower absolute binding agent application, due to which, again, the fire cargo applied by the usually organic binding agent, will be correspondingly reduced. Due to the reduction of the insulating weight, there will, at the same time, also be an advantageous reduction of the sustaining load of the channel, which is essentially important, especially in the cases of a freely suspended channel, since this sustaining force has to be statically collected.

In the cases of special geometry of a climate, respectively ventilation channel, it may be advantageous to utilize for the outer lining, wire mesh mats according to the invention, based on their flexibility with a binding agent content of <1 weight %. Wire mesh mats obtain their mechanical stability through a wire mesh, interlaced with the fiber structure, and therefore only a reduced content of binding agent is required, thus considerably reducing the overall fire load. Compared to wire mesh mats of conventional rock wool with comparable binding agent content, a considerable weight economy is of decisive importance.

On the other side, in the case of platelike insulating elements, a binding agent application in the range of 4,5 to 6 weight %, particularly 4,5 to 5,5% is preferably foreseen, in order to provide reinforced insulating elements, which reduce the danger of the so called “Mattress effect” when being used as internal linings. At the same time, protective action is being taken against a local fiber dissolution phenomenon, as a result of pulsation and twirling of a rapidly flowing agent, which is expressed by an advantageous rupture resistance.

At the same time, in view of the finely configured fiber structure, which is formed homogenously via the cross section of the insulating element, the essential portion of air for the insulating effect inside the insulating element, is being increased, which also results in a corresponding increase of the insulating effect in the cases of internal and external linings. Finally, in view of the finer configuration of the fibers, an advantageous λ-arithmetic value results according to DIN 18165 of ≦35 mW/mK, with simultaneous lower gross density.

This λ-arithmetic value may be advantageously realized in the cases of the outer linings with a fire resistance category L30 or similar, with gross densities between 20 and 40 kg/m³, preferably 30 kg/m³, with a fire resistance category L60 or similar with gross densities between 60 and 80 kg/m³, preferably 70 kg/m³ and a fire resistance category L90 or similar, with gross densities between 90 and 120 kg/m², preferably 110 kg/m³. In the case of inner linings, this λ-arithmetic value may be advantageously realized at least with a gross density corresponding to the gross density range of fire resistance category L30, and in order to preserve the technical sound protection demands, the insulating element of the invention offers a longitudinal flow resistance according to DIN EN ISO 9053 of >15 kPas/m². As far as it is referred to standards and examination requirements, reference is respectively made to the current version as filed on the filing date.

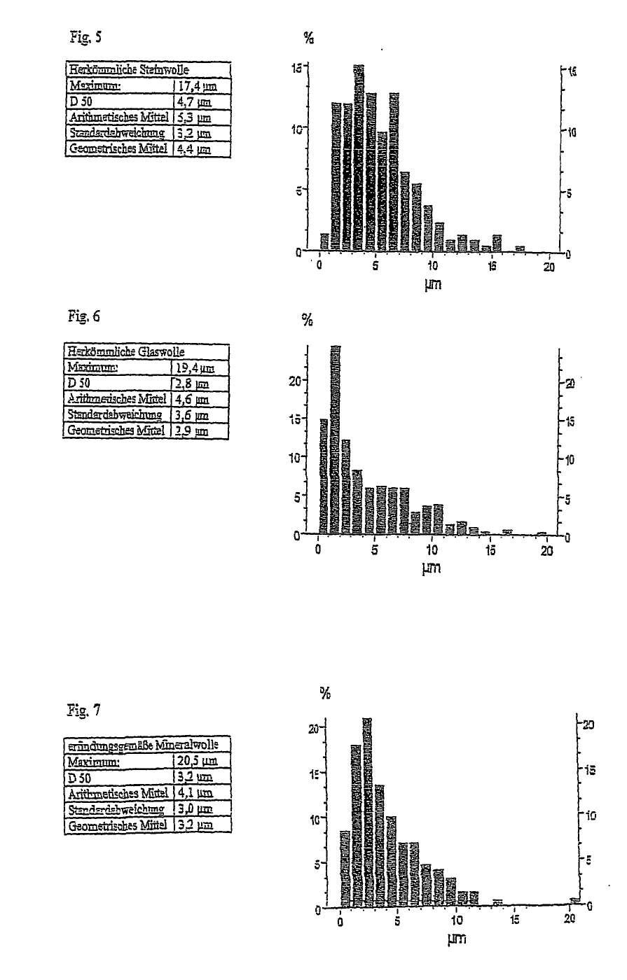

Especially preferred is a fiber fineness defined by an average geometric fiber diameter of 3 μm. The lower average geometric diameter, responsible for the fiber fineness is being determined based on the frequency distribution of the fiber diameter. The frequency distribution may be determined based on a wool test under the microscope. The diameter of a large number of fibers is being measure and recorded, resulting in an oblique left-sided distribution pattern (see FIGS. 5, 6 and 7).

It is finally convenient that in the event of utilization of the insulating element of the invention as internal lining, to provide a lamination for said element, which is an attrition-proof, acoustically transparent texture, such as a glass fleece, and in the case of an outer lining, with a diffusion-proof texture would be provided, such as an aluminum foil. Conveniently, the point of fusion of the insulating element according to the invention, is advantageously of ≧1.000° C. according to DIN 4102, Part 17.

In order to obtain an insulating element which meets the demands of sound, heat and fire protection in the range of climate, i.e. ventilation channels in a product, it will be convenient to utilize a glass composition, whose fusion at an internal centrifugation step pursuant to the basket centrifuging process, features a centrifuging basket temperature of 1.100° C. Correspondingly, the centrifuging basket must be formed in a temperature-resistant fashion. At the same time, a positively fine fiber structure is obtained, which, contrary to conventional rock wool, is practically exempt of beads, meaning the bead portion in the fiber structure is <1%.

Advantageously, said insulating elements are formed of mineral fibers, soluble in a physiological milieu, corresponding to the demands of the European Guideline 97/69/EG and/or the demands of the German Norm for Dangerous Substances, Section IV, No. 22, whereby absence of health dangers of the insulating elements will be insured during their production, processing, utilization and elimination.

Subsequently, Table 1 features a preferred composition of the mineral fibers of insulating elements according to the invention, in ranges in weight %.

TABLE 1

| | | | |
|---|---|---|---|
| Si0₂ | 39-55% | preferably | 39-52% |
| Al₂0₃ | 16-27% | preferably | 16-26% |
| CₐO | 6-20% | preferably | 8-18% |
| M_{g}O | 1-5% | preferably | 1-4.9% |
| Na₂O | 0-15% | preferably | 2-12% |
| K₂0 | 0-15% | preferably | 2-12% |
| R₂0(Na₂0 + K₂0) | 10-14.7% | preferably | 10-13.5% |
| P₂0₅ | 0-3% | especially | 0-2% |
| Fe₂0₃ (iron, total) | 1.5-15% | especially | 3.2-8% |
| B₂0₃ | 0-2% | preferably | 0-1% |
| Ti0₂ | 0-2% | preferably | 0.4-1% |
| Other | 0-2.0% | | |

A preferred smaller range of SiO₂ is 39-44%, particularly 40-43%. A preferred smaller range for CaO is 9,5-20%, particularly 10-18%.

The composition according to the invention relies on the combination of a high Al₂O₃-content, of between 16 and 27%, preferably greater than 17% and/or preferably less than 25%, for a sum of the network-forming elements—SiO₂ and Al₂O₃— of between 57 and 75%, preferably greater than 60% and/or preferably less than 72%, with a quantity of alkali metal (sodium and potassium) oxides (R₂O) that is relatively high but limited to between 10-14,7%, preferably 10 and 13,5%, with magnesia in an amount of at least 1%.

These compositions exhibit remarkably improved behaviour at very high temperature.

Preferably, Al₂O₃ is present in an amount of 17-25%, particularly 20-25%, in particular 21-24,5% and especially around 22-23 or 24% by weight.

Advantageously, good refractoriness may be obtained by adjusting the magnesia-content, especially to at least 1,5%, in particular 2% and preferably 2-5% and particularly preferably ≧2,5% or 3%. A high magnesia-content has a positive effect which opposes the lowering of viscosity and therefore prevents the material from sintering.

In case Al₂O₃ is present in an amount of at least 22% by weight, the amount of magnesia is preferably at least 1%, advantageously around 1-4%, preferably 1-2% and in particular 1,2-1,6%. The content of Al₂O₃ is preferably limited to 25% in order to preserve a sufficiently low liquidus temperature. When the content of Al₂O₃ is present in a lower amount of for example around 17-22%, the amount of magnesia is preferably at least 2%, especially around 2-5%.

Finally, with a view to provide packing with economy of space, it will be convenient to configure said insulating elements in such a form that they may be compressed, at least in a relation of 1:2, up to a maximum gross density of 50 kg/m³, and at least in a relation of 1:3, especially up to a maximum gross density of 30 kg/m³, without altering their specific profile.

Additionally, in view of the outstanding mechanical properties of said insulating elements according to the invention, with a comparably low portion of binding agent between 4, and particularly preferred between 4,5 to 7 weight %, to produce a climate, respectively ventilation channel in form on a self-sustaining construction, i.e. the unit being formed exclusively of platelike insulating elements, reinforced with binding agent. Advantageously, said insulating elements are a whole part of a plate which may be bent around folds, as described in claims of EP 0 791 791, EP 1 339 649 and U.S. Pat. No. 6,311,456, to which reference is now expressly being made.

It is convenient, to provide at the internal and external face of the channel formed in this way with a diffusion-proof cover, such as an aluminum foil or similar unit, and this cover also contributes quite importantly to the stability of the self-sustaining channel.

Due to the synergistically cooperating measures according to the invention, there results, thus, a climate, resp. ventilation channel, which, featuring a reduced thickness of the insulating elements and reduced weight as a consequence of reduced gross density, features low λ-arithmetic values, attending, in an advantageous fashion, the demands of sound, heat and fire protection in a product. As a result of reduced gross density, there results a low weight of the insulating element, with identical satisfactory insulating effect. As a result of the high degree of effectiveness of the binding agent, there also results a high rigidity, and as a result of the selected alkali/earth alkali mass relation of <1, the structure also distinguishes itself by a high temperature resistance. The bound fibers according to the invention offer a high mechanical elasticity and high temperature resistance, as compared to glass wool. The reduced gross density, added to the extraordinary high resistance, results thus in an insulating material of light weight, which is high of stable format and there may be assembled easily, i.e. exempt of fatigues factors. Especially, the insulating element of the invention features the same fire protection qualities as conventional rock wool, so that vis-à-vis the outstanding mechanical properties and reduced weight, also the full fire protection effect of conventional rock wool insulating elements are important. The invention creates thus a symbiosis between glass wool and rock wool and suitably combines their advantageous properties, with the insulating element being configured with fiber structure similar to glass wool, with identical high temperature resistance.

Subsequently the invention will be described in more detail, based on different examples of embodiments, with reference to the drawing. The figures show:

FIG. 1 partial section of the ventilation channel in rectangular format with schematically shown international insulation and external insulation,

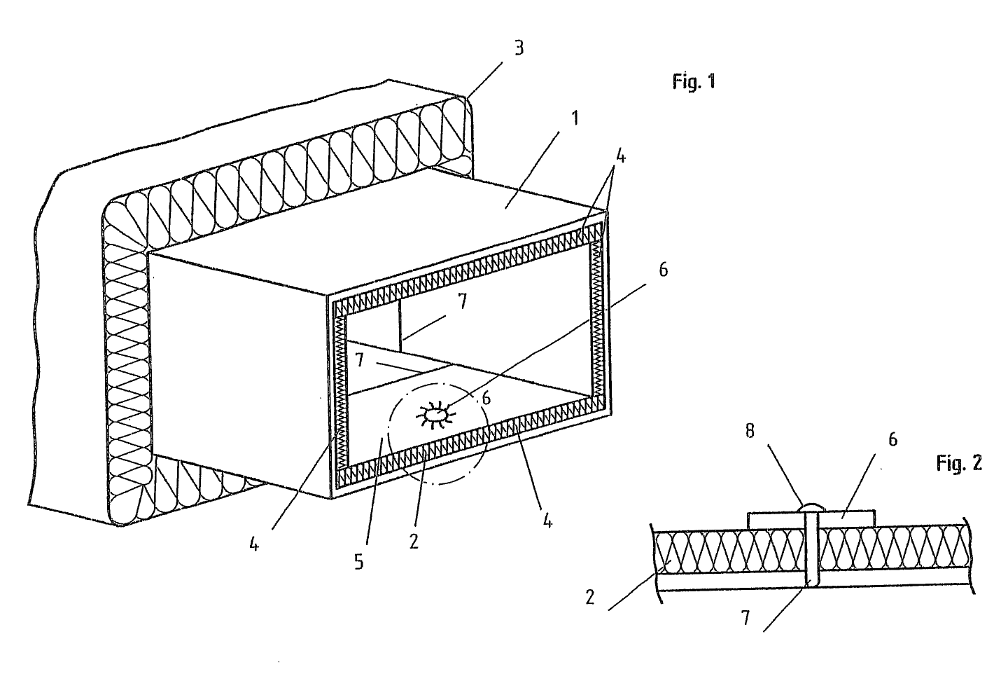

FIG. 2 a representation of a detail marked with a circle in FIG. 1, to exemplary explain the attachment of the lining and

FIG. 3 a simplified representation, in perspective, of a self-sustaining ventilation channel,

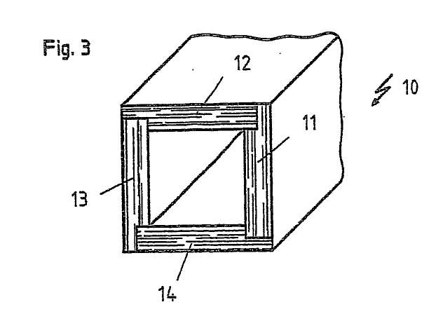

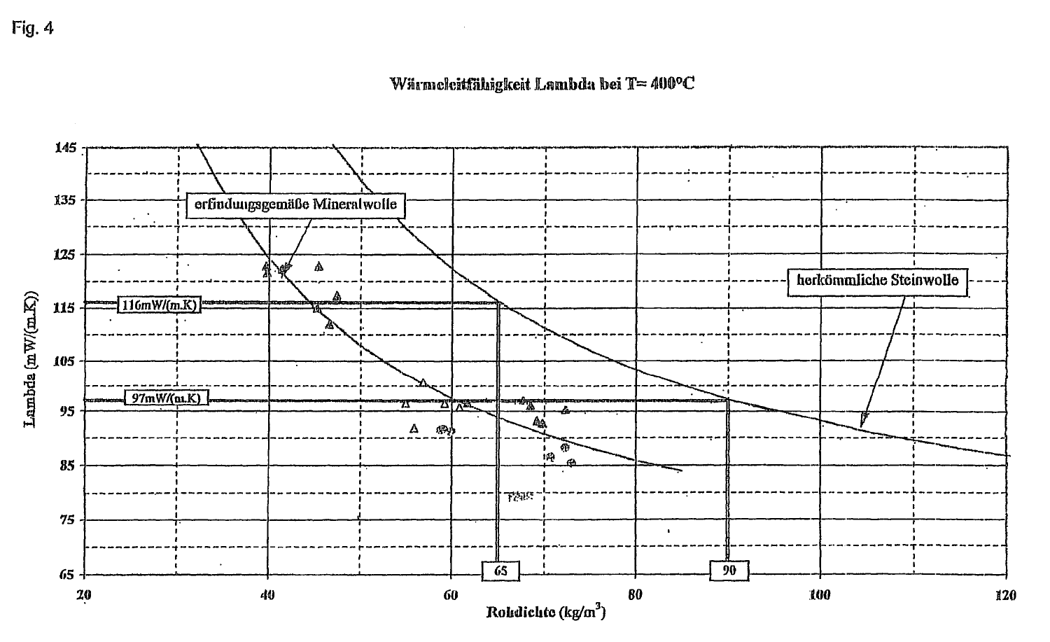

FIG. 4 a diagram of a comparative essay in the context of the heat conductivity test at 400° C.,

FIG. 5 a typical fiber-histogram of conventional rock wool,

FIG. 6 a typical fiber histogram of conventional glass wool, and

FIG. 7 a typical fiber histogram of mineral wool according to the invention.

FIG. 1 designates with number 1 a steel plate ventilation channel of rectangular transversal section. This channel is provided with an internal insulating, designated, as a whole with 2, and with an outer insulation, designated, as whole, with 3.

Said internal insulation 2 consists of platelike mineral wool insulating elements 4 with a lamination 5, for example of glass fleece, at the side of the internal insulation, turned towards the flux. The lamination protects the surface fibers and renders feasible a low-resistance flux of the flow medium.

In the shown exemplary embodiment, mineral wool insulating elements 4 feature a gross density of 30 kg/m³, with a weight of organic binding element in the form of phenol-formaldehyde resin of 5 weight % (dry, referring to the fiber mass). The average geometric fiber diameter is 3,2 μm, and the product features a λ-arithmetic value of 35 mW/mK, and with a longitudinal flow resistance of 17 kPas/m², features a thickness of 20 mm.

The fiber material of the platelike mineral wool insulating elements 4 is produced by internal centrifugation according to the centrifuging basket process, said elements being attached with retaining disks 6 at the wall of the conducting channel.

As a consequence of the high degree of efficiency of the binding agent of the phenol-formaldehyde resin exerted upon the fibers, and in view of the high mechanical elasticity of the individual fibers, there results a mineral wool insulating element, with a structure similar to a glass wool insulating element, also produced with internal centrifugation, however being considerably more resistant and rigid and which, in case of need, features a point of fusion above 1000° C. In this case, not only the lamination 5 is firmly retained at the surface of the insulating element 4 and there is no danger that it will be separated in the area of transversal junction 7 under the influence of pulsation and twirling of the eventually quickly flow agent. In addition, said retaining disks 6 generate the required retention force, without penetrating too extensively into the material, so that the so called “mattress effect”, negatively affecting a smooth flow wall, is being minimized, being principally excluded.

FIG. 2 features, in a merely schematic representation, details of the attachment of said internal insulation 2. For this purpose, on the ventilation channel 1, produced from steel plate, different pins 7 are disposed (only one being shown) and are here welded at the ventilation channel. It is also possible to glue the pins at the ventilation channel. The inner insulation is being pressed upon these pins and subsequently, from the upper section, i.e. from the inside of the ventilation channel, a retaining disk 6 is applied, which, in the present case, is fixed, i.e. attached over a threaded component 8, and alternately also a beat rivet is feasible to be applied. The light indenture of said internal insulation 2 at its internal surface is only designed to illustrate the so called “mattress effect”, which may be preset at conventional insulation, but which is widely avoided with the insulating plates according to the invention, as a consequence their rigid configuration.

The outer insulation 3, in the exemplary embodiment shown, is formed by a wire mesh mat, which, in conventional fashion, is externally attached to said ventilation channel 1 with a mat retaining hook or similar device, not shown here.

In the case of disposition in two layers of said external insulation 3, which is prescribed with the configurations corresponding to fire resistance categories L30, L60 or L90 according to DIN 4102, Part 4, the junctions of said insulating elements are disposed reciprocally offset in a form not shown in detail, so that flames, i.e. heat, may not project at an opening junction point until the plate cylinder of the ventilation channel 1. The wire mesh mat features, in the exemplary embodiment, the same parameters for gross density and average geometric fiber diameter as the internal insulation 2, and the organic binding agent portion in this case amounts only to 0,8 weight %.

Instead of a wire mesh mat for the external lining, it is also possible to produce the latter with individual platelike insulating elements, whose fiber structure is equivalent to the international insulation. Such platelike insulating elements possess the same gross density and thickness as the wire mesh mat, descried in the exemplary presentation, since both these parameters considerably influence the fire resistance.

Finally, FIG. 3 features in a simplified, schematical perspective representation a self-sustaining ventilation channel 10, composed of different insulating elements 11 through 14 at their junctions over folds with rectangular transversal section. Said insulating elements 11 through 14 consist of a glass composition according to Table 2 and are laminated with an aluminum foil at the inner and outer side, in such a way that said aluminum foil is disposed in circunferencial order at the outside.

The composition in weight 5% of the conventional insulating elements, produced from convention rock wool, as well as insulating elements, formed from convention glass wool, and the insulating elements according to the invention, can be seen in Table 2, and the conventional rock wool, as well as the insulating element according to the invention, feature a point of fusion of at least 1000° C. according to DIN 4102, Part 17.

TABLE 2

| | Conventional | Conventional | Insulating elements |
|---|---|---|---|
| Material | rock wool | glass wool | according to invention |
| SiO₂ | 57.2 | 65 | 41.2 |
| Al₂0₃ | 1.7 | 1.7 | 23.7 |
| Fe₂0₃ | 4.1 | 0.4 | 5.6 |
| TiO₂ | 0.3 | | 0.7 |
| CₐO | 22.8 | 7.8 | 14.4 |
| M_{g}O | 8.5 | 2.6 | 1.5 |
| Na₂0 | 4.6 | 16.4 | 5.4 |
| K₂0 | 0.8 | 0.6 | 5.2 |
| B₂0₃ | | 5 | |
| P₂0₅ | | 0.15 | 0.75 |
| MnO | | 0.3 | 0.6 |
| SrO | | | 0.5 |
| BaO | | | 0.34 |
| Total | 100 | 99.95 | 99.89 |

FIG. 4 features a measurement sequence of a thermal conductivity test at 400° C. over gross density in the form of a diagram. The measuring results were determined according to DIN 52612-1 with a so-called double-plate instrument.

It can be seen, in simple fashion, from this diagram which economy potential is feasible by utilizing the mineral wool according to the invention, as compared to conventional rock wool and, based on a example, for two gross densities of 65 and 90 kg/m³. The same thermal conducting capacity of 116 mW/mK, which is being attained with conventional rock wool with a gross density of 65 kg/m³, is reached with the mineral wool of the invention according with a gross density of approximately 45 kg/m³, i.e. with a weight economy of approximately 31%. In analog fashion, with a gross density of 90 kg/m³ of conventional rock wool, with the mineral wool of the invention there results a weight economy of approximately 33%.

Finally, FIGS. 5 and 6 indicate the conventional rock wool, mentioned in the description, and conventional glass wool, in a typical fiber histogram of the insulating elements, and FIG. 7 features a fiber histogram of the insulating elements according to the invention.

Finally, comparable essays in regard of insulating elements for ventilation channels were conducted, whereby respectively one insulating element made of mineral wool according to invention and indicated as IM is compared to an insulating element made of conventional rock wool. This applies for insulating elements in fire resistance categories L 30 (Table 1), L 60 (Table 2) and L 90 (Table 3).

TABLE 1

| | Requirement | Measured | Gross | Thick- | Surface | Loss due |
|---|---|---|---|---|---|---|
| Mate- | after | value nach | density | ness | weight | burning |
| rial | 30 min | 30 min | [kg/m³] | [mm] | [kg/m²] | [%] |
| Rock | <100 K | <100 K | 80 | 60 | 4.8 | 4 |
| wool | | | | | | |
| IM | <100 K | <100 K | 34 | 80 | 2.72 | 4.5 |

TABLE 2

| | Requirement | Measured | Gross | Thick- | Surface | Loss due |
|---|---|---|---|---|---|---|
| Mate- | after | value after | density | ness | weight | burning |
| rial | 60 min | 60 min | [kg/m³] | [mm] | [kg/m²] | [%] |
| Rock | <100 K | <100 K | 84 | 100 | 8.4 | 4 |
| wool | | | | | | |
| IM | <100 K | <100 K | 67 | 80 | 5.36 | 4.5 |

TABLE 3

| | Requirement | Measured | Gross | Thick- | Surface | Loss due |
|---|---|---|---|---|---|---|
| Mate- | after | value after | density | ness | weight | burning |
| rial | 90 min | 90 min | [kg/m³] | [mm] | [kg/m²] | [%] |
| Rock | <100 K | <100 K | 100 | 120 | 12 | 4 |
| wool | | | | | | |
| IM | <100 K | <100 K | 100 | 80 | 8 | 4.5 |

The requirement to be fulfilled by the essay examples is that after a firing test on one side of an insulating element within 30 min for L 30 respectively 60 min for L 60 respectively 90 min for L 90 no change in temperature >100 K occurs on the other side of the insulating element, meaning that the requirement is fulfilled, if the change in temperature is <100 K. As the table shows, all examples fulfill the requirement, whereby this results in significant differences in regard of the surface weight against insulating elements made of conventional rock wool, and in the case of table 1 and 2, the requirement is also fulfilled for the IM mineral wool according to invention at a significantly lower gross density and thickness.

1. Climate, respectively ventilating channel with an inner-and/or outer lining composed of at least one insulating element, especially with a temperature resistance to fulfill the demands of the normative fire resistance categories or similar norms, in form of a plate, reinforced with a binding agent, or a wire mesh plate, composed of mineral fibers, soluble in a physiological milieu, with said inner-and/or outer lining composed of different insulating elements, which may be disposed at least in one layer in the direction of the longitudinal axis of said climate, respectively ventilating channel, to which they are attached, characterized in that the composition of said mineral fibers of the insulating element features an alkali/earth alkali relation of <1 and the fibrous structure of said insulating element is determined by an average geometric fiber diameter of ≦4 μm, in the range of 20 to 120 kg/m³ and a portion of the binding agent, referred to the fiber mass of said insulating element, in form of a plate, is in the range of 4 to 7 weight % or in form of a wire mesh mat in the range of 0.5 to 1 weight %.

2. Channel according to claim 1, characterized in that said binding agent is an organic binding agent, such as phenol-formaldehyde resin.

3. Channel according to claim 1, characterized in that the portion of binding agent, referred to the fiber mass of said insulating element in the form of a plate, is within the range of 4.5 to 6 weight %.

4. Channel according to claim 1, characterized in that the gross density of the outer lining, in a fire resistance category L30 or similar category, amounts to 20 to 40 kg/m³, preferably 30 kg/m³, at a fire resistance category L60 or similar, it amounts to 60 to 80 kg/m³, preferably 70 kg/m³, and at a fire resistance category L90 or similar, it amounts to 90 to 120 kg/m³, preferably 110 kg/m³.

5. Channel according to claim 1, characterized in that said gross density of the inner lining corresponds, at least, to a gross density of fire resistance category L30 or similar, featuring a longitudinal flux resistance according to DIN EN ISO 9053 or >15 kPas/m².

6. Channel according to claim 1, characterized in that said insulating element features an λ-arithmetic value of ≦35 mW/mK.

7. Channel according to claim 1, characterized in that said inner lining consists of an attrition-resistant, acoustically transparent cover, such as a glass fleece or similar device, and said outer lining is laminated of a diffusion-resistant cover, such as an aluminum foil or similar unit.

8. Channel according to claim 1, characterized in that said insulating element features a point of fusion according to DIN 4102, Part 17, of ≧1.000° C.

9. Channel according to claim 1, characterized in that said mineral fibers of the insulating element are produced by internal centrifugation pursuant to the centrifuging basket with a centrifuging basket temperature of at least 1,000° C.

10. Channel according to claim 1, characterized in that said mineral fibers of the insulating element, concerning their solubility in a physiological milieu, meet the demands of the European Guideline 97/69/EG and/or the demands of the German Norm for Dangerous Products, Section IV, No. 22.

11. Channel according to claim 1, characterized in that said insulating elements, for packing purposes, may be compressed at least in a relation of 1:2, up to a maximum gross density of 50 kg/m³, especially at least in a relation of 1:3 up to a maximum gross density of 30 kg/m³.

12. Inner-/outer lining for a climate, respectively ventilating channel, characterized in that said inner-/outer lining is composed of at least an insulating element with the marking features of claim 1.

13. Inner-/outer lining according to claim 11, characterized by the following ranges of the chemical composition of said mineral fibers in weight % figures:

| | |
|---|---|
| Si0₂ | 39-55% |
| A1₂0₃ | 16-27% |
| CₐO | 6-20% |
| M_{g}O | 1-5% |
| Na₂O | 0-15% |
| K₂0 | 0-15% |
| R₂0(Na₂0 + K₂0) | 10-14.7% |
| P₂0₅ | 0-3% |
| Fe₂0₃ (iron, total) | 1.5-15% |
| B₂0₃ | 0-2% |
| Ti0₂ | 0-2% |
| Other | 0-2.0% |

14. Self-sustaining climate, respectively ventilating channel according to claim 1, characterized in that it is exclusively composed of platelike insulating elements, reinforced with binding agent, said elements featuring a lamination on their inner- and outer surface.

15. Channel according to claim 14, characterized in that said inner-and outer lamination is formed of a diffusion-resistant cover, such as an aluminum foil or similar device.

16. Channel according to claim 14, characterized in that the insulating elements are connected together at their junctions via folds to a rectangular respectively square cross-section.
